(19) United States
(12) Patent Application Publication
(10) Pub. No.: US 2021/0393186 A1
(43) Pub. Date: Dec. 23, 2021

(54) VISUALIZATION PROGRAM, VISUALIZATION METHOD, AND VISUALIZATION SYSTEM

(71) Applicant: FUJITSU LIMITED, Kawasaki-shi (JP)

(72) Inventor: Masahiro WATANABE, Kawasaki (JP)

(73) Assignee: FUJITSU LIMITED, Kawasaki-shi (JP)

(21) Appl. No.: 17/315,354

(22) Filed: May 10, 2021

(30) Foreign Application Priority Data
Jun. 18, 2020 (JP) 2020-105184

(51) Int. Cl. A61B 5/343; A61B 5/346; G16H 50/20

(52) U.S. Cl. CPC A61B 5/343; G16H 50/20; A61B 5/346

(57) ABSTRACT

A computer-based visualization method includes: arranging, based on electrocardiogram data that indicates an electrocardiogram obtained by sensing from a patient in a predetermined period, a plurality of curves in time series in an axial direction perpendicular to a time axis and a potential axis of the electrocardiogram, each of the plurality of curves indicates a partial electrocardiogram corresponding to a respective beat from among the electrocardiogram indicated in the electrocardiogram data; generating a curved surface that includes the plurality of arranged curves; generating a plane that intersects the curved surface; and outputting a figure based on an intersection line between the curved surface and the plane.

### CROSS-REFERENCE TO RELATED APPLICATION

This application is based upon and claims the benefit of priority of the prior Japanese Patent Application No. 2020-105184, filed on Jun. 18, 2020, the entire contents of which are incorporated herein by reference.

### FIELD

The embodiments discussed herein are related to a visualization program, a visualization method, and a visualization system.

### BACKGROUND

An electrocardiogram is used to find a disease of a heart. The electrocardiogram is obtained by graphing an electrical activity of the heart. The electrical activity of the heart may be measured by an electrocardiograph. The electrocardiograph is capable of recording an electrocardiogram as digital data (hereinafter, referred to as electrocardiogram data). A doctor may find a disease of a patient at an early stage by visualizing a state of a heart by a computer based on electrocardiogram data of the patient.

For example, a computer may simulate a motion of a three-dimensional model of a patient's heart based on electrocardiogram data, and reproduce a movement of the patient's heart with the three-dimensional model on the computer. Visualization of the movement of the heart makes it easier to find a behavior caused by a heart disease.

As a visualization technique for medical data, for example, an ultrasonic diagnostic apparatus has been proposed that is capable of acquiring diagnosis information representing a blood flow volume from an ultrasonic Doppler spectrum image as uniform data more easily and in a short time. A physiological mapping data display method including dynamically displaying an electrocardiogram data set corresponding to an electrical activity detected by one or more electrodes on a display unit has also been proposed. Further, there has also been proposed a portable electrocardiograph having an enlargement/reduction display function so that a plurality of types of trend graphs and histograms may be reproduced and displayed.

Examples of the related art include Japanese Laid-open Patent Publication No. 2010-200844, Japanese National Publication of International Patent Application No. 2017-512113, and Japanese Laid-open Patent Publication No. 04-300523.

### SUMMARY

According to an aspect of the embodiments, there is provided a computer-based visualization method. In an example, the method includes: arranging, based on electrocardiogram data that indicates an electrocardiogram obtained by sensing from a patient in a predetermined period, a plurality of curves in time series in an axial direction perpendicular to a time axis and a potential axis of the electrocardiogram, each of the plurality of curves indicates a partial electrocardiogram corresponding to a respective beat from among the electrocardiogram indicated in the electrocardiogram data; generating a curved surface that includes the plurality of arranged curves; generating a plane that intersects the curved surface; and outputting a figure based on an intersection line between the curved surface and the plane.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 illustrates an example of a visualization method according to a first embodiment;

FIG. 2 illustrates an example of a visualization system according to a second embodiment;

FIG. 3 illustrates an example of hardware of a visualization system;

FIG. 4 is a block diagram illustrating functions of a visualization system;

FIG. 5 illustrates an example of electrocardiogram data;

FIG. 6 illustrates an example of heart model data;

FIG. 7 illustrates an example of a space in which an electrocardiogram is arranged;

FIG. 8 illustrates a first example of an electrocardiogram for each beat in a case where there is a disease;

FIG. 9 illustrates a second example of an electrocardiogram for each beat in a case where there is a disease;

FIG. 10 illustrates a first example of a cross section obtained based on a plurality of curves indicating an electrocardiogram for each beat;

FIG. 11 illustrates a second example of a cross section obtained based on a plurality of curves indicating an electrocardiogram for each beat;

FIG. 12 illustrates a third example of a cross section obtained based on a plurality of curves indicating an electrocardiogram for each beat;

FIG. 13 illustrates a fourth example of a cross section obtained based on a plurality of curves indicating an electrocardiogram for each beat;

FIG. 14 illustrates a first example of generation of a curved surface based on an electrocardiogram;

FIG. 15 illustrates an example of a cross-sectional view when a symptom of Brugada syndrome appears;

FIG. 16 illustrates a second example of generation of a curved surface based on an electrocardiogram;

FIG. 17 illustrates an example of a cross-sectional view when a symptom of tachycardia appears;

FIG. 18 illustrates an example of a change in a cross-sectional view due to a movement of a cutting plane;

FIG. 19 illustrates a first example indicating an expression pattern of a disease;

FIG. 20 illustrates a second example indicating an expression pattern of a disease;

FIG. 21 is a flowchart illustrating an example of a procedure of visualization processing; and

FIG. 22 illustrates an example of a visualization screen.

### DESCRIPTION OF EMBODIMENT(S)

It may take time to find out a disease appearing in an electrocardiogram simply by displaying a graph of the electrocardiogram. For example, there is a case where a patient carries a small-sized electrocardiograph and acquires electrocardiogram data of the patient for 24 hours or longer. For example, that is a case where an unnatural behavior of a heart due to a disease of a patient intermittently appears, or a case where a symptom of the disease is expressed only when the patient is in a specific state. In these cases, a presence of such a disease may be found out by checking an electrocardiogram based on long-term electrocardiogram data. However, even when an electrocardiogram for a long time is displayed as it is, it takes time for a doctor to visually check the electrocardiogram.

According to an aspect of the embodiments, there is provided a solution to enable visualization of an influence of a disease appearing in an electrocardiogram in an easily understandable manner.

Hereinafter, the embodiments will be described with reference to the drawings. Note that each of the embodiments may be implemented by combining a plurality of embodiments in a range not causing any contradiction.

### First Embodiment

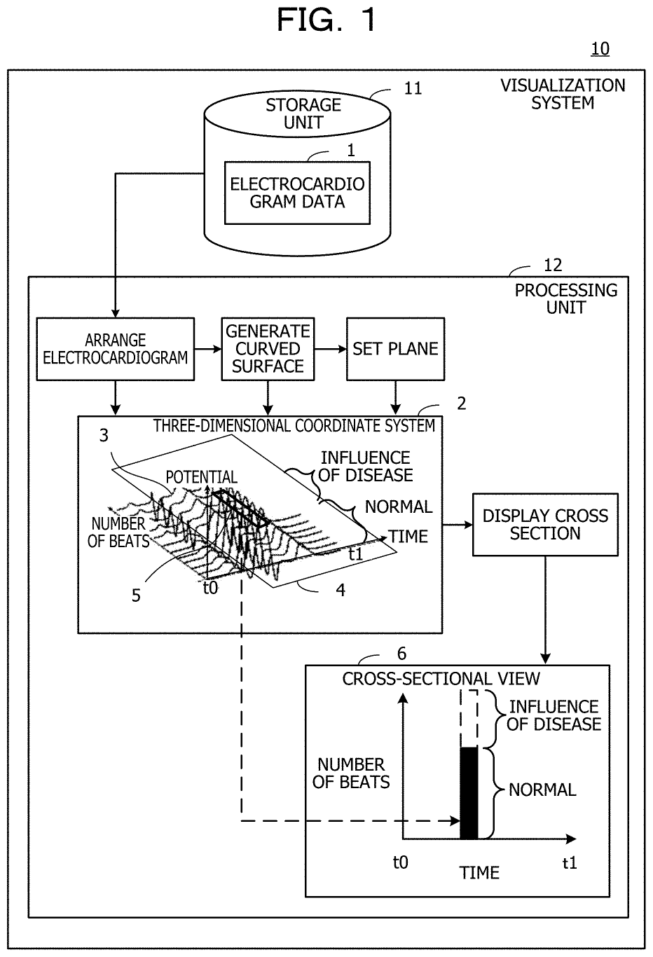

FIG. 1 illustrates an example of a visualization method according to a first embodiment. FIG. 1 illustrates a visualization system 10 for implementing a visualization method of an electrocardiogram. The visualization system 10 may implement a visualization method by executing a visualization program in which a processing procedure of the visualization method is described, for example.

The visualization system 10 may be realized by one or a plurality of computers. The visualization system 10 also includes a storage device coupled to the computer. The visualization system 10 includes a storage unit 11 and a processing unit 12. The storage unit 11 is, for example, a memory or a storage device of the computer. The processing unit 12 is, for example, a processor or an arithmetic circuit of the computer.

The storage unit 11 stores electrocardiogram data 1. The electrocardiogram data 1 is data indicating an electrocardiogram of the patient for a predetermined period. An electrocardiogram is a graph indicating an electrical activity of a patient's heart. The graph of the electrocardiogram is represented on a plane having a time axis and a potential axis.

The processing unit 12 visualizes an influence of a disease appearing in the electrocardiogram based on the electrocardiogram data 1 in an easily understandable manner. That is, based on the electrocardiogram data 1 indicating the electrocardiogram of the patient in the predetermined period, the processing unit 12 arranges a plurality of curves 3 indicating the electrocardiogram of each beat in a three-dimensional coordinate system 2 in time series in an axial direction perpendicular to the time axis and the potential axis of the electrocardiogram. Next, the processing unit 12 generates a curved surface including the plurality of arranged curves 3. Further, the processing unit 12 generates a plane 4 intersecting the generated curved surface. Then, the processing unit 12 outputs a figure based on an intersection line 5 between the generated curved surface and the plane 4. For example, the processing unit 12 outputs a cross-sectional view 6 indicating a cross section when a region having a lower potential than the curved surface is cut by the plane 4 based on the intersection line 5 between the curved surface and the plane 4. The cross-sectional view 6 is displayed, for example, on a monitor.

Note that the plane 4 is, for example, a plane parallel to the time axis and the number-of-beats axis. In a case where the plane 4 is parallel to the time axis and the number-of-beats axis, the plane 4 is a plane of a specific potential. Then, the intersection line 5 between the curved surface and the plane 4 corresponds to an equipotential line of the potential indicated by the plane 4. In a case where the plane 4 is parallel to the time axis and the number-of-beats axis, in the cross-sectional view 6, a figure obtained by filling an inside of the intersection line 5 in the plane 4 is displayed as a cross section when a region having a lower potential than the curved surface is cut by the plane 4.

Further, the plane 4 may be a plane parallel to the number-of-beats axis, and inclined with respect to the time axis. In a case where the plane 4 is inclined with respect to the time axis, for example, a figure obtained by filling an inside of the figure obtained by projecting the intersection line 5 to a plane parallel to the time axis and the number-of-beats axis with a color different from the color of the other portion is displayed as a cross section when a region having a lower potential than the curved surface is cut by the plane 4.

In this manner, in a case where the influence of the disease appears in a part of the electrocardiogram recorded in the electrocardiogram data 1 for a long period, it becomes possible to easily recognize that the symptom of the disease appears. For example, it is assumed that, when a symptom of a disease appears in a patient, an intensity (potential) of an R wave of the electrocardiogram becomes weak. In a case where such a disease is expected, for example, a user instructs the visualization system 10 to generate the plane 4 intersecting the curved surface including the plurality of curves 3 indicating an electrocardiogram for each beat at a position slightly below a peak of the R wave of the electrocardiogram at the normal time. Then, the processing unit 12 generates the plane 4 at the designated position and outputs the cross-sectional view 6 based on the intersection line 5 at that time. In the cross-sectional view 6, a cross section is displayed during a period (a range in the direction of the number-of-beats axis) in which a symptom of a disease does not appear in the patient, but the cross section is not displayed during other periods. By referring to the cross-sectional view 6, the user may easily determine that the symptom of the disease appears in the patient during a period in which the cross section is not displayed.

The processing unit 12 may also move the position of the generated plane 4 parallel to the direction of the potential axis of the electrocardiogram. In this case, every time the plane 4 moves, the processing unit 12 outputs a figure based on the intersection line 5 between the curved surface and the plane 4. For example, the processing unit 12 repeatedly moves the plane 4 at regular intervals, and displays the cross-sectional view 6 that corresponds to the position of the plane 4 on the monitor each time. Accordingly, even in a case where an appropriate position of the plane 4 is unknown in advance, it is possible to easily recognize a period in which the symptom of the disease appears by the change in the cross-sectional shape displayed in the cross-sectional view 6 due to the movement of the plane 4.

In the arrangement of the plurality of curves 3, the processing unit 12 may normalize a time taken for one beat such that a time taken for one beat indicated in the electrocardiogram of each beat becomes a predetermined time and arrange the plurality of curves 3 indicating the electrocardiogram of each beat. By normalizing the time taken for one beat, it is possible to suppress that the electrocardiogram becomes difficult to read due to the influence of the variation of the heart rate of the patient.

Note that when the time taken for one beat is not normalized, the influence of the disease may become clear in some cases. For example, as in a case of a bradycardia, in a case where the shapes (waveforms) of a plurality of curves indicating the electrocardiogram of one beat are not changed significantly from those in the normal state, but the heart rate changes significantly, the influence of the disease appears clearly in the electrocardiogram when the time taken for one beat is not normalized.

### Second Embodiment

Next, a second embodiment will be described. The second embodiment is a visualization system that visualizes a portion where an influence of a disease appears in electrocardiogram data in an easily understandable manner, and visualizes a state of the heart due to the influence of the disease by performing a heart simulation based on the electrocardiogram data of the portion.

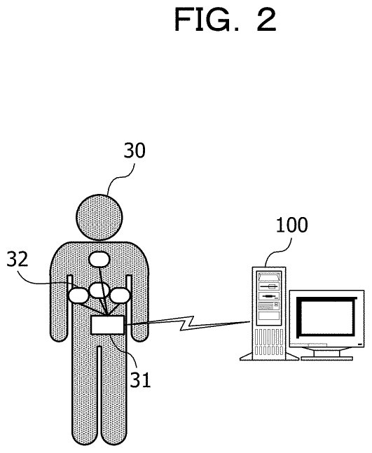

FIG. 2 illustrates an example of a visualization system according to the second embodiment. A patient 30 wears, for example, a portable electrocardiograph 31. The portable electrocardiograph 31 is also referred to as a Holter electrocardiograph. A plurality of electrodes 32 are coupled to the electrocardiograph 31. Each of the plurality of electrodes 32 is attached to a predetermined portion of a chest of the patient 30. The electrocardiograph 31 measures an electric signal transmitted through the heart via the plurality of electrodes 32 and records the electric signal as electrocardiogram data. For example, the electrocardiograph 31 records electrocardiogram data for a period of 24 hours or longer. The electrocardiograph 31 may communicate with a visualization system 100 wirelessly or by wire.

The visualization system 100 acquires electrocardiogram data from the electrocardiograph 31. Then, the visualization system 100 visualizes the state of the heart of the patient 30 based on the acquired electrocardiogram data.

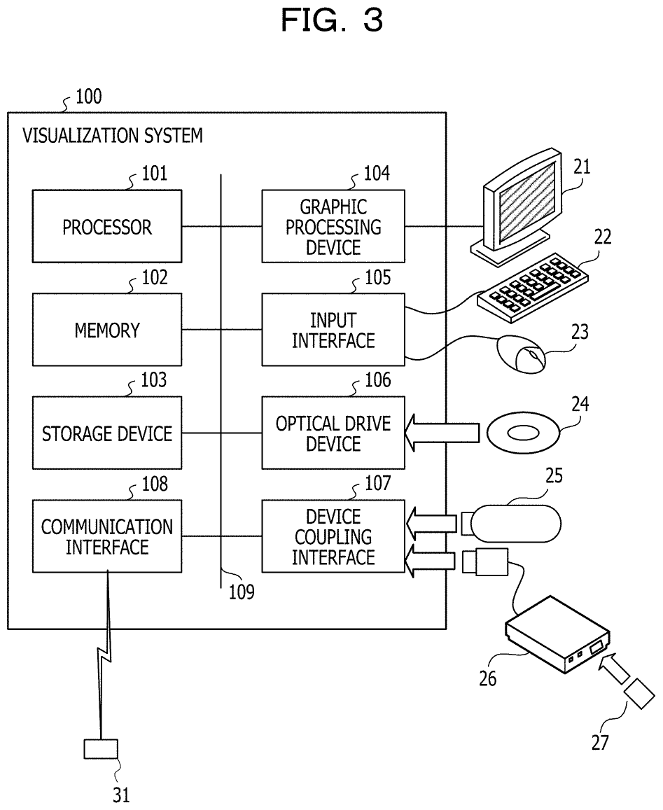

FIG. 3 illustrates an example of hardware of the visualization system. The visualization system 100 is entirely controlled by a processor 101. A memory 102 and a plurality of peripheral devices are coupled to the processor 101 via a bus 109. The processor 101 may be a multiprocessor. The processor 101 is, for example, a central processing unit (CPU), a microprocessor unit (MPU), or a digital signal processor (DSP). At least a part of functions implemented by the processor 101 executing a program may be implemented by an electronic circuit such as an application-specific integrated circuit (ASIC) or a programmable logic device (PLD).

The memory 102 is used as a main storage device of the visualization system 100. The memory 102 temporarily stores at least some of programs of an operating system (OS) and application programs to be executed by the processor 101. Further, the memory 102 stores various pieces of data used in processing performed by the processor 101. As the memory 102, for example, a volatile semiconductor storage device such as a random-access memory (RAM) is used.

Examples of the peripheral devices coupled to the bus 109 include a storage device 103, a graphic processing device 104, an input interface 105, an optical drive device 106, a device coupling interface 107, and a communication interface 108.

The storage device 103 electrically or magnetically writes and reads data to and from a recording medium built therein. The storage device 103 is used as an auxiliary storage device of a computer. The storage device 103 stores the programs of the OS, the application programs, and the various pieces of data. Note that as the storage device 103, for example, a hard disk drive (HDD) or a solid-state drive (SSD) may be used.

A monitor 21 is coupled to the graphic processing device 104. The graphic processing device 104 causes an image to be displayed on a screen of the monitor 21 in accordance with an instruction from the processor 101. Examples of the monitor 21 include a display device using an organic electroluminescence (EL), a liquid crystal display device, or the like.

A keyboard 22 and a mouse 23 are coupled to the input interface 105. The input interface 105 transmits, to the processor 101, signals transmitted from the keyboard 22 and the mouse 23. Note that the mouse 23 is an example of a pointing device, and other pointing devices may also be used. The other pointing devices may be a touch panel, a tablet, a touch pad, a trackball, or the like.

The optical drive device 106 reads data recorded on an optical disc 24 or writes data to the optical disc 24 by using a laser beam or the like. The optical disc 24 is a portable recording medium on which data is recorded such that the data is readable through reflection of light. The optical disc 24 may be a Digital Versatile Disc (DVD), a DVD-RAM, a compact disc read-only memory (CD-ROM), a CD-recordable (CD-R), a CD-rewritable (CD-RW), or the like.

The device coupling interface 107 is a communication interface for coupling the peripheral devices to the visualization system 100. For example, a memory device 25 and a memory reader/writer 26 may be coupled to the device coupling interface 107. The memory device 25 is a recording medium having a communication function of communicating with the device coupling interface 107. The memory reader/writer 26 is a device that writes data to a memory card 27 or reads data from the memory card 27. The memory card 27 is a card-type recording medium.

The communication interface 108 transmits and receives data to and from other computers or communication devices. The communication interface 108 may be a wireless communication interface that is wirelessly coupled to and communicates with a wireless communication device such as a base station or an access point by radio. The communication interface 108 wirelessly communicates with the electrocardiograph 31, for example.

The hardware described above enables the visualization system 100 to implement processing functions according to the second embodiment. Note that, the visualization system 10 described in the first embodiment may also be implemented by the same hardware as that of the visualization system 100 illustrated in FIG. 3.

The visualization system 100 realizes the processing functions in the second embodiment by, for example, executing the program recorded on a computer-readable recording medium. The program in which content of processing to be executed by the visualization system 100 is described may be recorded on various recording media. For example, the program to be executed by the visualization system 100 may be stored in the storage device 103. The processor 101 loads at least a part of the program in the storage device 103 into the memory 102 and executes the program. Further, the program to be executed by the visualization system 100 may be recorded on a portable recording medium such as the optical disc 24, the memory device 25, or the memory card 27. The program stored in the portable recording medium may be installed in the storage device 103 and executed under the control of the processor 101, for example. Further, the processor 101 may read the program directly from the portable recording medium and execute the program.

By using the visualization system 100 having such hardware, it is possible to visualize the influence of the disease appearing in the electrocardiogram based on the electrocardiogram data acquired from the electrocardiograph 31. For example, the visualization system 100 visualizes a portion where the influence of the disease of the patient 30 appears in the electrocardiogram so as to easily specify the portion. Moreover, the visualization system 100 performs a heart simulation by using the electrocardiogram data of the portion where the influence of the disease of the patient 30 appears, and reproduces the behavior of the heart when the influence of the disease appears with a three-dimensional model.

Note that when a waveform appearing in the electrocardiogram is simply displayed on the screen as it is, the following problem occurs.

In a case where a service for reproducing the behavior of the heart of the patient 30 is to be realized by the heart simulation based on electrocardiogram data, the doctor first specifies a portion where the influence of the disease appears from the entire electrocardiogram data. When the electrocardiogram data of the patient 30 is long-term data of 24 hours or longer, it takes time for a doctor to visually check the waveform of the entire electrocardiogram and specify a portion where the influence of the disease appears.

Therefore, the visualization system 100 visualizes the electrocardiogram so that a unique portion in the electrocardiogram is clarified in order to shorten the time for specifying the data to be input to the heart simulation in the electrocardiogram data. However, it is difficult to specify the unique portion from the electrocardiogram in the following points. 1) It is not easy to appropriately read the state of the heart from the electrocardiogram of the changing portion (not only the corresponding electrocardiogram but also the surrounding electrocardiogram). 2) It is difficult to display an actual time interval of one beat in an easily understandable manner. 3) It is difficult to extract a sinus bradycardia with a heart rate of 50 beats per minute (bpm) or less or sinus tachycardia with a heart rate of 100 beats per minute (bpm) or more due to the influence of the sinus rhythm. 4) It is not easy to correctly distinguish a difference between features of waveforms for respective diseases.

Note that examples of the diseases that may be distinguished from the electrocardiogram include arrhythmias and abnormalities in excitation conduction. There are various types of arrhythmias. For example, there are various symptoms of arrhythmia such as atrial tachycardia, ventricular tachycardia, and atrioventricular block. Since there are many types of diseases of which an influence appears in the electrocardiogram, in a case where a doctor simply checks only a waveform of the electrocardiogram, it is not easy to quickly determine what kind of disease causes an abnormality of the electrocardiogram even though the abnormality may be found.

In particular, there is a case where the influence of the disease does not appear all the time on the electrocardiogram, and appears only a few times a day. In such a case, an electrocardiogram of the patient 30 is measured for a long period of time by using the portable electrocardiograph 31 as illustrated in FIG. 2. Then, the electrocardiogram to be checked by the doctor is also an electrocardiogram for a long period of time, and it takes time to specify a portion where the influence of the disease appears therefrom.

Therefore, the visualization system 100 visualizes the electrocardiogram so that a portion where the influence of the disease appears can be easily specified even in the electrocardiogram for a long period of time.

Functions of the visualization system 100 will be described below.

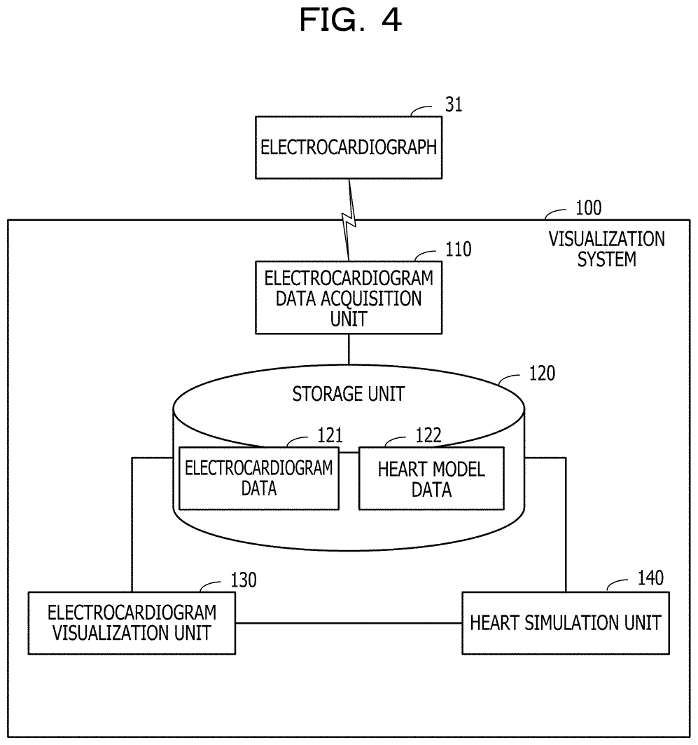

FIG. 4 is a block diagram illustrating functions of the visualization system. The visualization system 100 includes an electrocardiogram data acquisition unit 110, a storage unit 120, an electrocardiogram visualization unit 130, and a heart simulation unit 140.

The electrocardiogram data acquisition unit 110 acquires electrocardiogram data 121 from the electrocardiograph 31. The electrocardiogram data acquisition unit 110 stores the acquired electrocardiogram data 121 in the storage unit 120. The electrocardiogram data acquisition unit 110 is realized, for example, by the processor 101 controlling the communication interface 108.

The storage unit 120 stores the electrocardiogram data 121 and heart model data 122. The heart model data 122 is data of a three-dimensional model (heart model) representing the shape of the heart of the patient 30. The storage unit 120 is implemented by using, for example, a part of a storage area of the memory 102 or the storage device 103 of the visualization system 100.

The electrocardiogram visualization unit 130 visualizes the electrocardiogram based on the electrocardiogram data 121. For example, the electrocardiogram visualization unit 130 divides the electrocardiogram data 121 into data for each beat of the heartbeat, and generates a graph in which a plurality of curves indicating the electrocardiogram corresponding to the divided data are arranged.

Further, the electrocardiogram visualization unit 130 generates a curved surface including the plurality of arranged curves indicating the electrocardiogram, and displays a cross-sectional view obtained by cutting the curved surface by a predetermined plane. In a case where the influence of the disease appears in the electrocardiogram for a limited period of time, the cross-sectional shape of the curved surface indicated in the cross-sectional view becomes a distorted shape, and it becomes clear at a glance that there is a disease. The electrocardiogram visualization unit 130 transmits the electrocardiogram data of the portion where the disease is detected to the heart simulation unit 140. The electrocardiogram visualization unit 130 may be realized, for example, by causing the processor 101 to execute a visualization program for electrocardiogram visualization.

The heart simulation unit 140 simulates the behavior of the heart during a period in which the disease appears by using the electrocardiogram data of the portion where the disease is detected. For example, the heart simulation unit 140 reads the heart model data 122 from the storage unit 120. Next, the heart simulation unit 140 generates a heart model of the patient 30 from the heart model data 122. Then, the heart simulation unit 140 deforms the shape of the heart model according to the electrocardiogram data of the portion where the disease is detected, and displays the change of the heart model on the monitor 21. The heart simulation unit 140 may be realized, for example, by causing the processor 101 to execute a heart simulation program for a heart simulation.

Next, the electrocardiogram data 121 will be described.

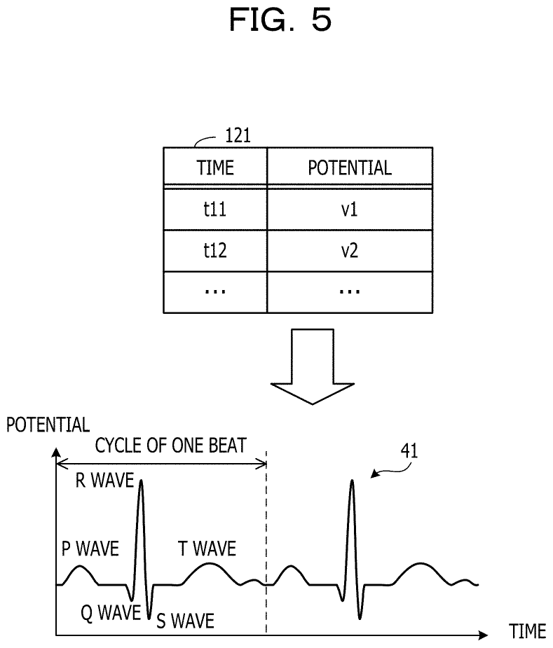

FIG. 5 illustrates an example of the electrocardiogram data. In the electrocardiogram data 121, potentials measured by the electrodes are set in association with the measurement time. An electrocardiogram 41 may be generated from the electrocardiogram data 121. In the electrocardiogram 41, a horizontal axis represents a time and a vertical axis represents a potential.

In the electrocardiogram 41, similar waveforms are repeated for each cycle of the heartbeat. Within one cycle of beating, waveforms called a P wave, a Q wave, an R wave, an S wave, and a T wave are mainly included. Hereinafter, the P wave, the Q wave, the R wave, the S wave, and the T wave may be collectively referred to as a PQRST wave. The doctor determines the presence or absence of the disease based on a shape, a width, and a height of these waves. Further, the time intervals between the plurality of waves are also used to determine the presence or absence of the disease.

Next, the heart model data 122 will be described.

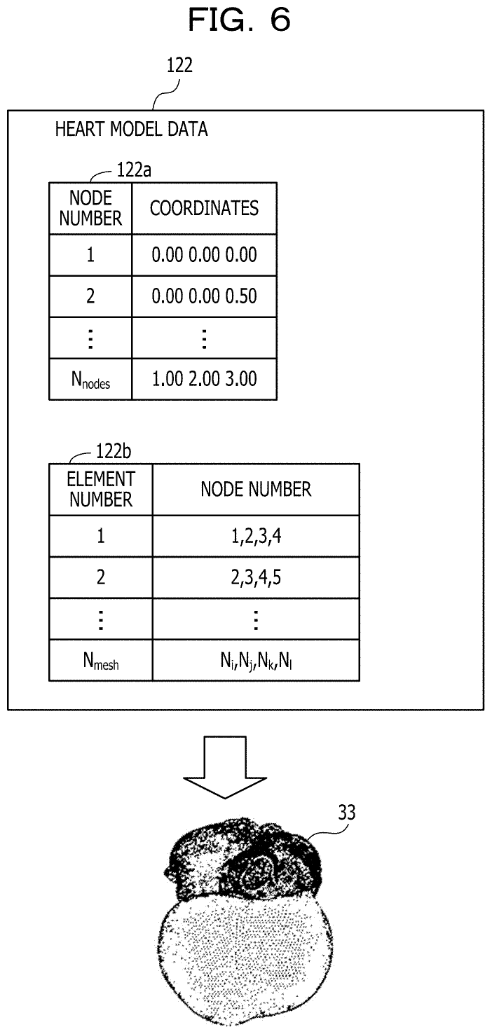

FIG. 6 illustrates an example of the heart model data. The heart model data 122 is, for example, unstructured grid data. The heart model data 122 includes a node information table 122a and an element information table 122b. In the node information table 122a, a node number and coordinates indicating a position of a node are set for each node. Note that the coordinates of each node set in the node information table 122a indicate a position of the node before a simulation starts, and the position of the node changes when the heartbeat is reproduced by the simulation. In the element information table 122b, an element number and node numbers of the nodes that are vertices of a tetrahedral element are set for each element.

Based on such heart model data 122, a three-dimensional heart model 33 may be generated. The heart model 33 is a collection of the tetrahedral elements. By giving the condition of the electric signal based on the electrocardiogram data to such a heart model 33, it is possible to execute a reproduction simulation of a propagation state (excitation propagation) of the electric signal through the myocardium of the heart. Then, by reproducing the contraction and expansion movements of the myocardium with the heart model 33 according to the state of the excitation propagation, it is possible to visualize the movements of the actual heart of the patient 30.

Next, the processing in the electrocardiogram visualization unit 130 will be specifically described. The electrocardiogram visualization unit 130 first divides the electrocardiogram data 121 into data for each beat. Then, the electrocardiogram visualization unit 130 arranges the electrocardiogram for one beat in a three-dimensional coordinate system by aligning the time of each electrocardiogram for one beat, for example.

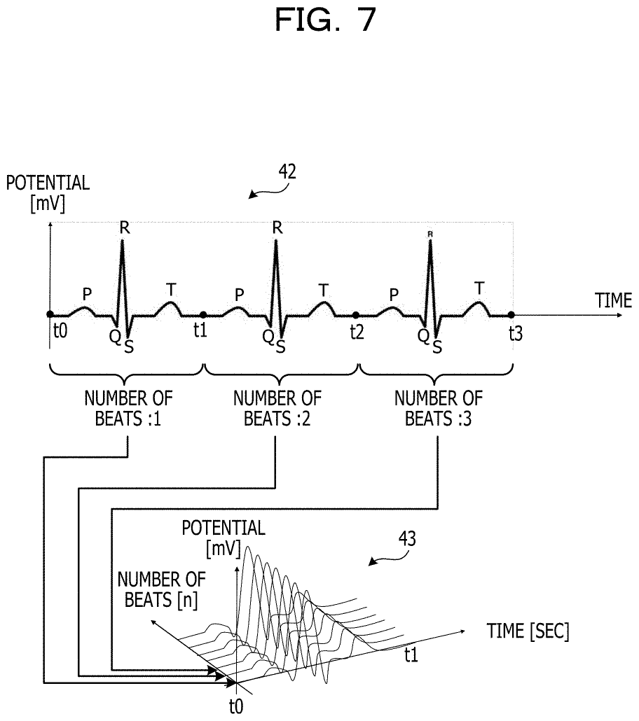

FIG. 7 illustrates an example of a space in which an electrocardiogram is arranged. For example, the electrocardiogram visualization unit 130 detects a PQRST wave from a time-series change in a potential indicated in an electrocardiogram 42, and sets a start time of the P wave as a division time. Then, the electrocardiogram visualization unit 130 groups, for each time zone with the division time as a separation time, records of electrocardiogram data in which the time within the corresponding time zone is set. In the example of FIG. 7, the electrocardiogram data is divided into a time zone of time t0 to time t1, a time zone of time t1 to time t2, and a time zone of time t2 to time t3. The time zone of time t0 to time t1 includes electrocardiogram data representing an electrocardiogram of a first beat (number of beats: 1). The time zone of time t1 to time t2 includes electrocardiogram data representing an electrocardiogram of a second beat (number of beats: 2). The time zone of time t2 to time t3 includes electrocardiogram data representing an electrocardiogram of a third beat (number of beats: 3).

The electrocardiogram visualization unit 130 arranges the electrocardiogram for one beat based on the each of the electrocardiogram data for each beat in a three-dimensional coordinate system 43 with the start time aligned with t0. The three-dimensional coordinate system 43 has a time [sec] axis, a potential [mV] axis, and the number-of-beats [n] axis. The electrocardiogram 42 illustrated in FIG. 7 indicates an electrocardiogram when the heart beats normally. Therefore, the waveforms indicating the electrocardiogram for each beat are also uniform.

Note that even in people without heart disease, a speed of pulse is not uniform. For example, the speed of pulse differs between when ascending stairs and when seated in a chair. When the speed of the pulse is fast, a cycle of one beat is short. In a case of comparing electrocardiogram waveforms for each beat, it is difficult to compare the waveforms when the cycle of one beat varies. Therefore, the electrocardiogram visualization unit 130 is capable of normalizing the beating time for each beat, for example, in accordance with a length of the period of t0 (beating start time) to t1 (beating end time). The length of the period of t0 to t1 is, for example, an average cycle of the heartbeat in the normal state.

In a case of normalization, the electrocardiogram visualization unit 130 corrects the time of the measurement time of each potential from the start of the cycle of one beat in accordance with the cycle of one beat of t0 to t1. For example, it is assumed that the cycle of one beat of t0 to t1 is T1, and the cycle of one beat in the electrocardiogram data for one beat to be normalized is T2. At this time, the electrocardiogram visualization unit 130 multiplies an elapsed time of the measurement time of each potential for one beat to be normalized from the start of the beating by “T1/T2”. For example, in a case where the cycle T2 of one beat is half of the normal cycle T1 (T2=T1/2), the electrocardiogram visualization unit 130 multiplies the elapsed time from the start of the one beat to the measurement of each potential by two (T1/T2=T1/(T1/2)=2). In this way, the cycle of one beat may be matched with t0 to t1.

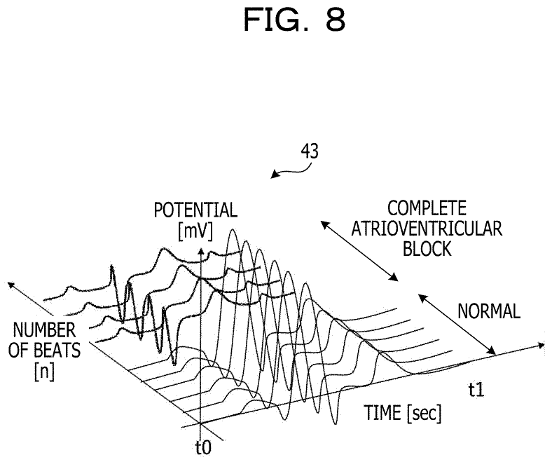

FIG. 8 illustrates a first example of an electrocardiogram for each beat in a case where there is a disease. FIG. 8 illustrates an example in which an electrocardiogram in a case where the patient 30 suffers from a complete atrioventricular block is divided and arranged in the three-dimensional coordinate system 43. In the example of FIG. 8, the cycle of each beat is normalized. In the complete atrioventricular block, an electric signal from an atrium side is not transmitted to a ventricle. Then, the ventricle generates an electric signal by itself and starts a contraction activity. As a result, the waveforms of the electrocardiogram are disordered.

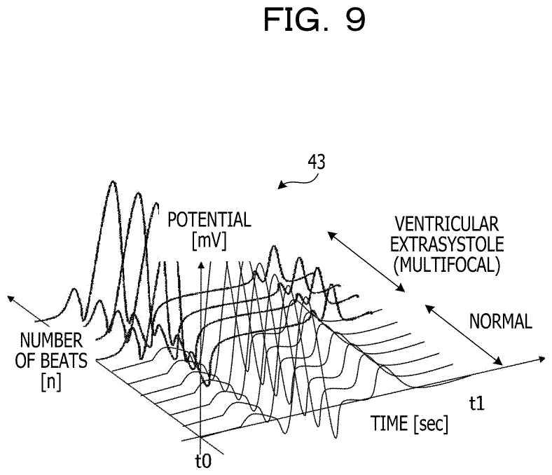

FIG. 9 illustrates a second example of the electrocardiogram for each beat in a case where there is a disease. FIG. 9 is a diagram illustrating an example in which an electrocardiogram in a case where the patient 30 suffers from multifocal ventricular extrasystole is divided and arranged in the three-dimensional coordinate system 43. In the example of FIG. 9, the cycle of each beat is normalized. In the ventricular extrasystole, an abnormal electrical stimulation generated in the ventricle activates the ventricle before a normal beat of the ventricle occurs. As a result, extra beats are generated in the ventricle, and the waveforms of the electrocardiogram are disordered.

In FIG. 8 or FIG. 9, since a portion where the influence of the disease appears is focused and illustrated, the influence of the disease is easily understood. However, it takes time to find out the waveform of the electrocardiogram affected by the disease as illustrated in FIG. 8 or FIG. 9 from the electrocardiogram for 24 hours, for example, by simply comparing the waveforms for each beat.

Therefore, the electrocardiogram visualization unit 130 generates a smooth curved surface including a plurality of curves indicating the electrocardiogram for each beat and arranged in the three-dimensional coordinate system 43. In the electrocardiogram of the normal heart as illustrated in FIG. 8, the generated curved surface is not inclined in the direction of the number-of-beats axis, and is a curved surface inclined only in the time axis direction. On the other hand, in a case where there is a disease, the waveforms of the electrocardiogram for each beat are not uniform.

In the example of FIG. 8 or FIG. 9, the smooth curved surface including the plurality of curves indicating the electrocardiogram for each beat arranged in the three-dimensional coordinate system 43 is largely distorted in the direction of the number-of-beats axis between the normal period and the period when the symptom of the complete atrioventricular block appears.

It may be seen that there is an electrocardiogram for each beat deviated from a normal state depending on the presence or absence of the inclination in the direction of the number-of-beats axis, but the electrocardiogram is to be observed in more detail to determine whether or not it is caused by a disease. Therefore, the electrocardiogram visualization unit 130 defines a plane for cutting a curved surface including a plurality of curves indicating the electrocardiogram in the three-dimensional coordinate system 43. The cutting plane is, for example, a plane parallel to the time axis and the number-of-beats axis. Further, the cutting plane is parallel to the number-of-beats axis, but may be a plane having a predetermined inclination with respect to the time axis.

The electrocardiogram visualization unit 130 obtains a cross section of a portion where a region on a lower side (in a negative direction of the potential axis) of the curved surface including the plurality of curves indicating the electrocardiogram for each beat and the cutting plane intersect. The electrocardiogram visualization unit 130 displays a cross-sectional shape on the monitor 21. A change in the waveform of the electrocardiogram caused by the disease clearly appears in the cross section.

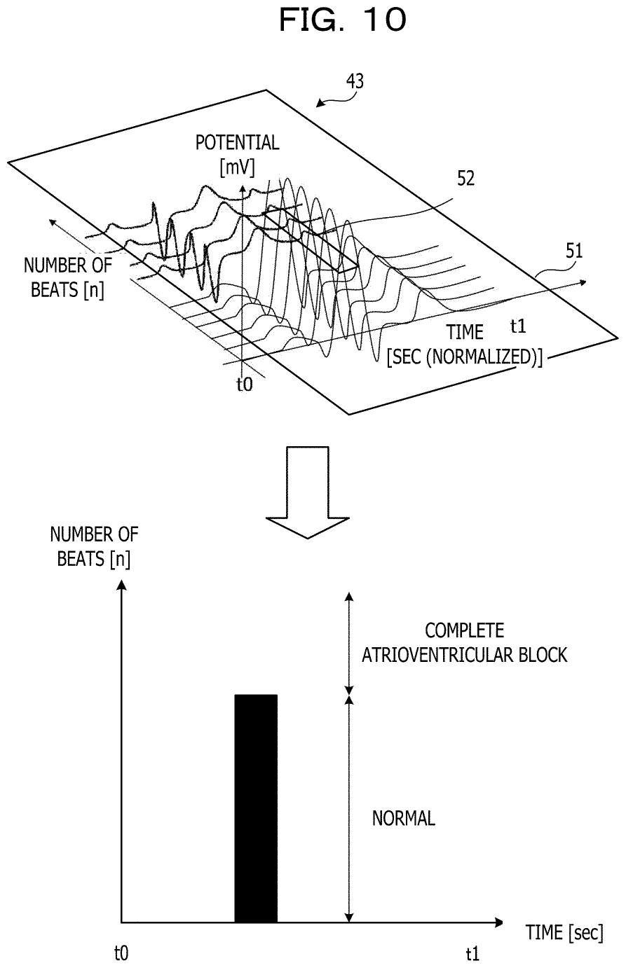

FIG. 10 illustrates a first example of the cross section obtained based on the plurality of curves indicating the electrocardiogram for each beat. FIG. 10 illustrates an example in which a region on the lower side of a curved surface including a plurality of curves indicating an electrocardiogram in which a symptom of complete atrioventricular block appears is cut by a cutting plane 51 to obtain a cross section 52 of the region on the lower side of the curved surface. Note that in the example of FIG. 10, the cycle of each beat is normalized.

When the symptom of the complete atrioventricular block appears, the height of the peak of the R wave becomes low (the potential at the peak of the R wave decreases). Therefore, when the cutting plane 51 is set at a position slightly lower than the height of the R wave (the potential at the peak of the R wave) in the normal case, the cross section does not appear during a period in which the symptom of the complete atrioventricular block appears, and it becomes clear that there is a disease of the complete atrioventricular block.

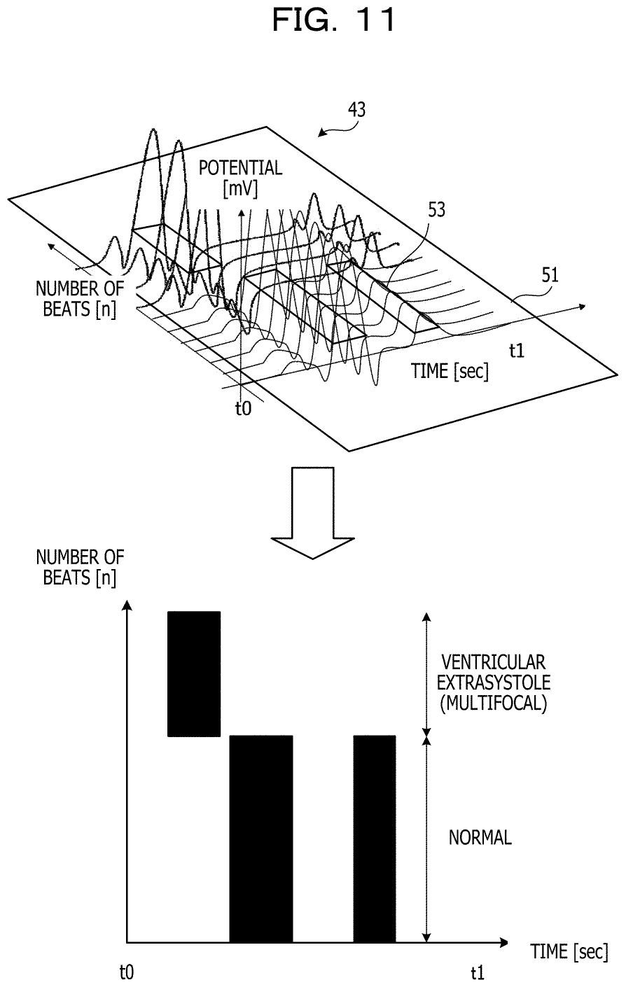

FIG. 11 illustrates a second example of a cross section obtained based on a plurality of curves indicating an electrocardiogram for each beat. FIG. 11 illustrates an example in which a region on the lower side of a curved surface including a plurality of curves indicating an electrocardiogram in which a symptom of a multifocal ventricular extrasystole appears is cut by the cutting plane 51 to obtain a cross section 53 of the region on the lower side of the curved surface. Note that in the example of FIG. 11, the cycle of each beat is normalized.

In the period in which the symptom of the multifocal ventricular extrasystole appears, an occurrence time of the R wave in one beat is shifted from the normal time. Further, during a period in which the symptom of the multifocal ventricular extrasystole appears, the height of the peak of the T wave is low. In this case, when the cutting plane 51 is set at a position slightly lower than the height (the potential at the peak of the T wave) of the T wave in the normal case, a period in which the symptom of the multifocal ventricular extrasystole appears becomes clear. That is, for example, since the position of the R wave is shifted and the cross section of the T wave does not appear, it becomes clear that the symptom of the multifocal ventricular extrasystole appears in the corresponding period.

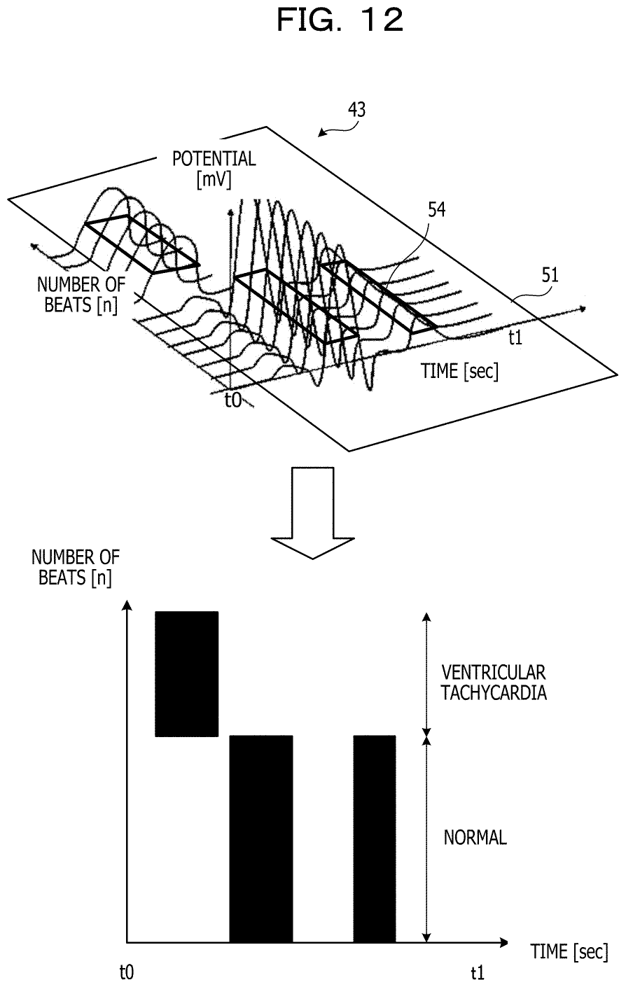

FIG. 12 illustrates a third example of a cross section obtained based on a plurality of curves indicating an electrocardiogram for each beat. FIG. 12 illustrates an example in which a region on the lower side of a curved surface including a plurality of curves indicating an electrocardiogram in which a symptom of ventricular tachycardia appears is cut by the cutting plane 51 to obtain a cross section 54 of the region on the lower side of the curved surface. Note that in the example of FIG. 12, the cycle of each beat is not normalized.

When the symptom of ventricular tachycardia appears, low-peak waveforms are repeated in a short cycle. Therefore, when the cutting plane 51 is set at a position slightly lower than the height (the potential at the peak of the T wave) of the T wave in the normal case, the cross section appears at an early stage during a period in which the symptom of the ventricular tachycardia appears, and the R wave and the T wave are not distinguished from each other, and thus it becomes clear that there is a disease of the ventricular tachycardia.

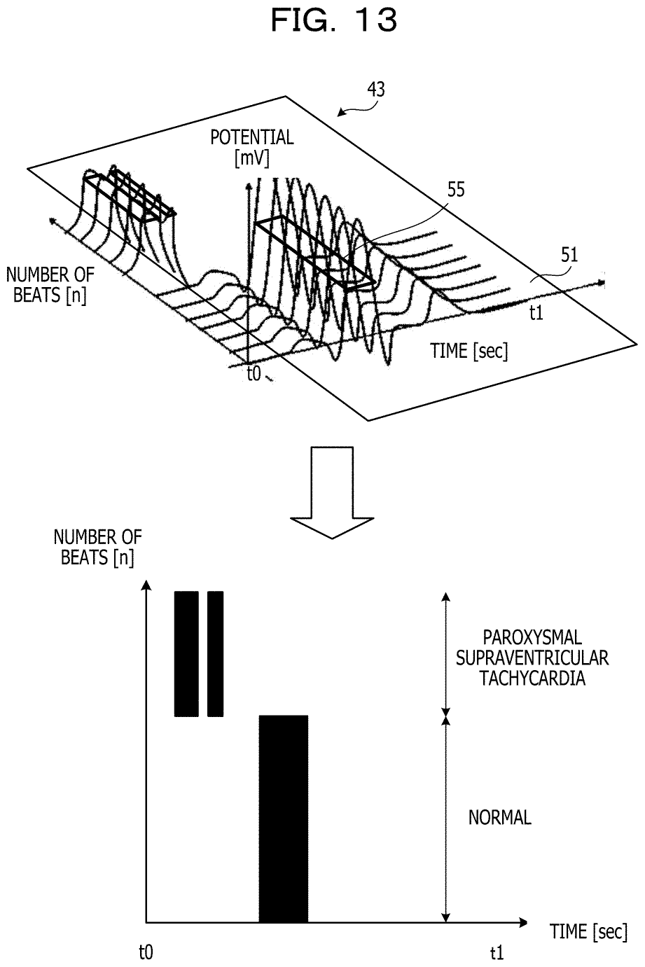

FIG. 13 illustrates a fourth example of a cross section obtained based on a plurality of curves indicating an electrocardiogram for each beat. FIG. 13 illustrates an example in which a region on the lower side of a curved surface including a plurality of curves indicating an electrocardiogram in which a symptom of paroxysmal supraventricular tachycardia appears is cut by the cutting plane 51 to obtain a cross section 55 of the region on the lower side of the curved surface. Note that in the example of FIG. 13, the cycle of each beat is not normalized.

When the symptom of the paroxysmal supraventricular tachycardia appears, waveforms of a mountain with two peaks are repeated in a short cycle. Therefore, when the cutting plane 51 is set at a position slightly lower than the height (the potential at the peak of the R wave) of the R wave in the normal case, during a period in which the symptom of the ventricular tachycardia appears, two cross sections divided into short periods appear at an early stage, and thus it becomes clear that there is a disease of paroxysmal supraventricular tachycardia.

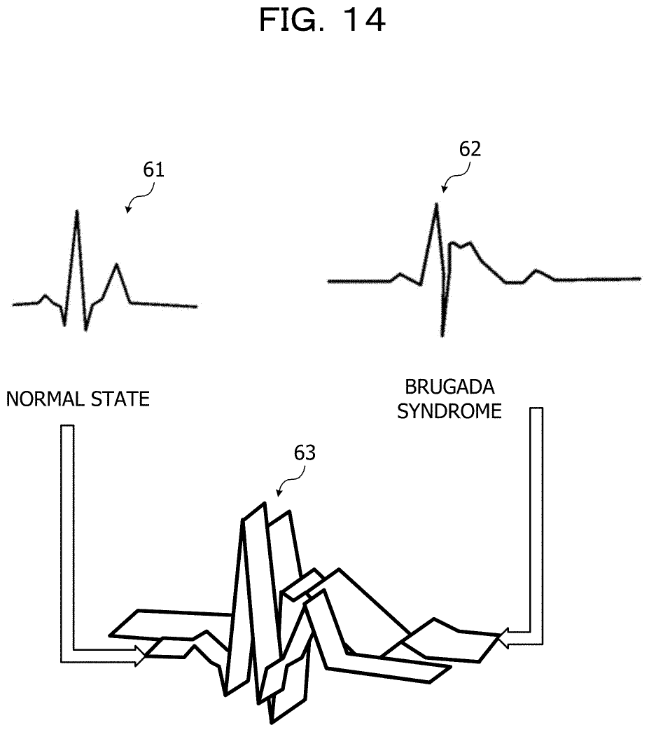

FIG. 14 illustrates a first example of generation of a curved surface based on an electrocardiogram. In the example of FIG. 14, an electrocardiogram 61 in a normal state and an electrocardiogram 62 when a symptom of Brugada syndrome appears are illustrated. A curved surface 63 is generated from each of the electrocardiograms 61 and 62. A cross-sectional view is generated by cutting the region on the lower side of the curved surface 63 by a cutting plane.

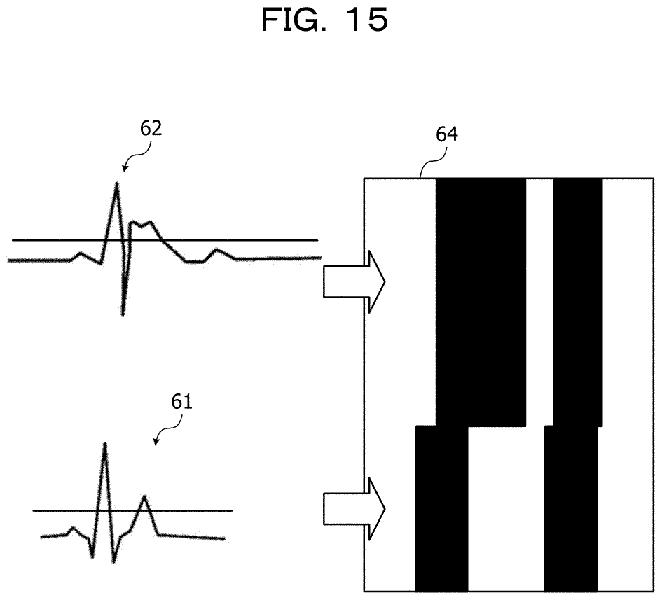

FIG. 15 illustrates an example of a cross-sectional view when a symptom of Brugada syndrome appears. In the example of FIG. 15, the cutting plane is set at a position passing slightly below the peak of the T wave. The electrocardiogram visualization unit 130 cuts the curved surface 63 by the cutting plane to generate a cross-sectional view 64. A cross section corresponding to the plurality of curves indicating the electrocardiogram 61 at the normal time is indicated on a lower side of the cross-sectional view 64, and a cross section corresponding to the plurality of curves indicating the electrocardiogram 62 when the symptom of the Brugada syndrome appears is indicated on an upper side of the cross-sectional view 64. The cross-sectional view 64 makes it clear that the symptom of the Brugada syndrome appears.

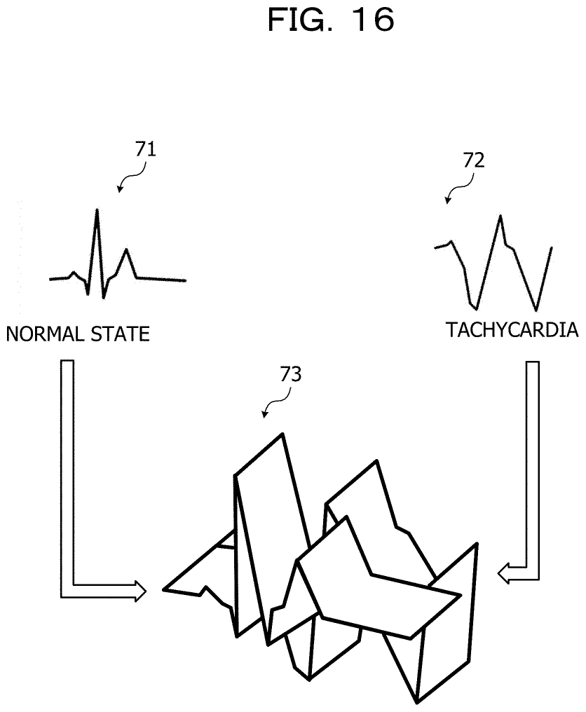

FIG. 16 illustrates a second example of generation of a curved surface based on an electrocardiogram. In the example of FIG. 16, an electrocardiogram 71 in a normal state and an electrocardiogram 72 when a symptom of tachycardia appears are illustrated. A curved surface 73 is generated from each of the electrocardiograms 71 and 72. The cross-sectional view is generated by cutting the curved surface 73 by the cutting plane.

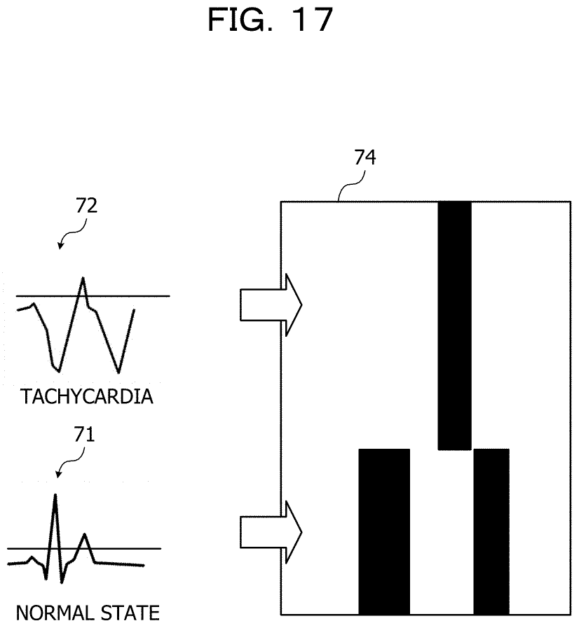

FIG. 17 illustrates an example of a cross-sectional view when a symptom of tachycardia appears. In the example of FIG. 17, the cutting plane is set at a position passing slightly below the peak of the T wave in the normal state. The electrocardiogram visualization unit 130 cuts the curved surface 73 by the cutting plane to generate a cross-sectional view 74. A cross section corresponding to the plurality of curves of the electrocardiogram 71 at the normal time is indicated on a lower side of the cross-sectional view 74, and a cross section corresponding to the plurality of curves of the electrocardiogram 72 when the symptom of the tachycardia appears is indicated on an upper side of the cross-sectional view 74. The cross-sectional view 74 makes it clear that the symptom of the tachycardia appears.

As described above, a cross-sectional view corresponding to a disease is generated. When such a cross-sectional view is displayed, changes in the electrocardiogram over a long period of time may be visually and easily recognized by changes in the shape of the cross-sectional view. Note that depending on the type of the disease of the patient, the position of the cutting plane at which the feature of the disease appears in the cross-sectional view is different. Therefore, the electrocardiogram visualization unit 130 gradually lowers the cutting plane from the upper side (in a positive direction of the potential axis), and displays the cross-sectional view for each position of the cutting plane. The doctor observes the change in the cross-sectional view due to the movement of the cutting plane, and checks whether or not the feature of the disease appears in the cross-sectional view.

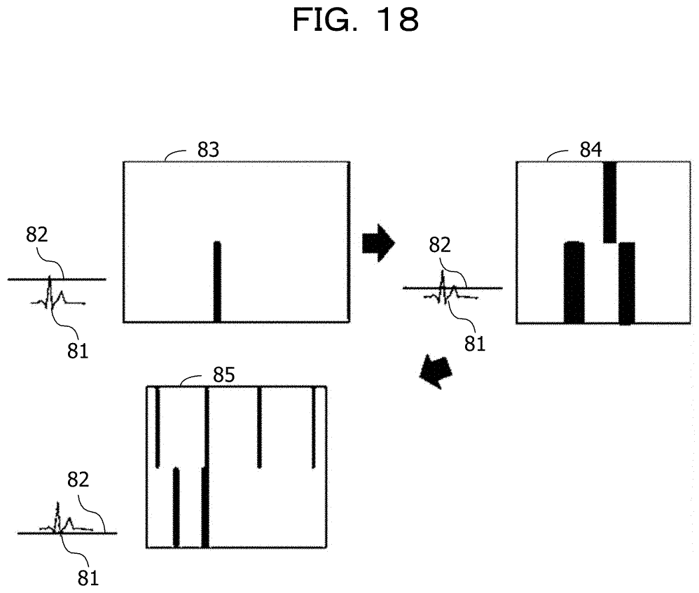

FIG. 18 illustrates an example of a change in a cross-sectional view due to the movement of the cutting plane. An electrocardiogram 81 illustrated in FIG. 18 indicates an electrocardiogram in the normal state. When a cutting plane 82 is gradually lowered from the upper side, a cross section in the vicinity of the peak of the R wave of the normal electrocardiogram 81 appears in a first cross-sectional view 83. In the cross-sectional view 83, since the cross section indicating the R wave is interrupted in the middle, it may be seen that a symptom of some disease appears. However, the type of disease may not be specified in the cross-sectional view 83.

When the cutting plane 82 moves to the lower side, for the normal period, cross sections of the period of the R wave and the vicinity of the peak of the T wave appear in a cross-sectional view 84. The cross section also appears during a period in which the influence of the disease appears.

When the cutting plane 82 further moves to the lower side, for the normal period, cross sections of the periods of the Q wave and the S wave appear in a cross-sectional view 85. During a period in which the influence of the disease appears, short-term cross sections appear at four locations.

As described above, by moving the position of the cutting plane 82, a state of the cross section during a period in which the influence of the disease appears changes. Then, the doctor may specify the type of the disease from the state of the change in the cross section.

By representing the change in the electrocardiogram by a cross-sectional view, it is possible to easily visualize the entire electrocardiogram for a long period of time. Visualization of the electrocardiogram for a long period of time makes it easy to grasp an expression pattern of the disease.

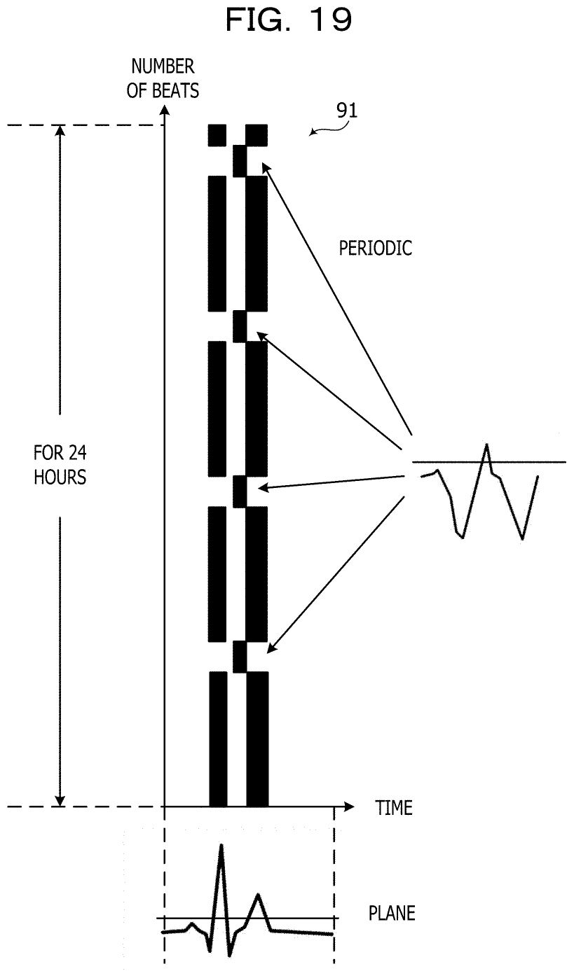

FIG. 19 illustrates a first example indicating an expression pattern of a disease. FIG. 19 illustrates a cross-sectional view 91 generated based on electrocardiogram data for 24 hours. Referring to the cross-sectional view 91, it is seen that a disorder of the electrocardiogram due to the disease periodically occurs in 24 hours. As described above, by visualizing the electrocardiogram in the cross-sectional view 91, it is easy to grasp that the expression of the symptom of the disease is periodic.

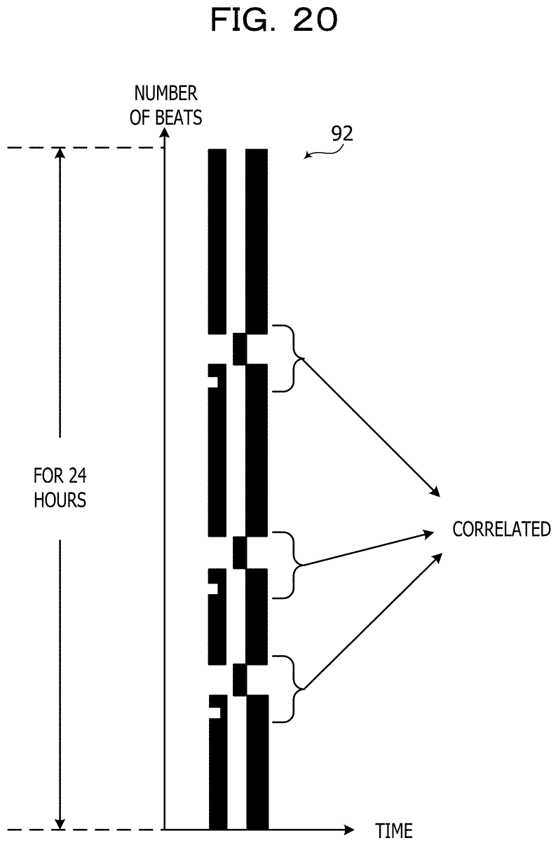

FIG. 20 illustrates a second example indicating an expression pattern of a disease. FIG. 20 illustrates a cross-sectional view 92 generated based on electrocardiogram data for 24 hours. Referring to the cross-sectional view 92, it may be seen that the disorder of the electrocardiogram due to the disease occurs after a specific disorder of the electrocardiogram occurs several times in 24 hours. As described above, by visualizing the electrocardiogram in the cross-sectional view 92, it is possible to easily grasp that the expression of the symptom of the disease is correlated with some other events that affect the electrocardiogram.

Next, a procedure of visualization processing in the visualization system 100 will be specifically described.

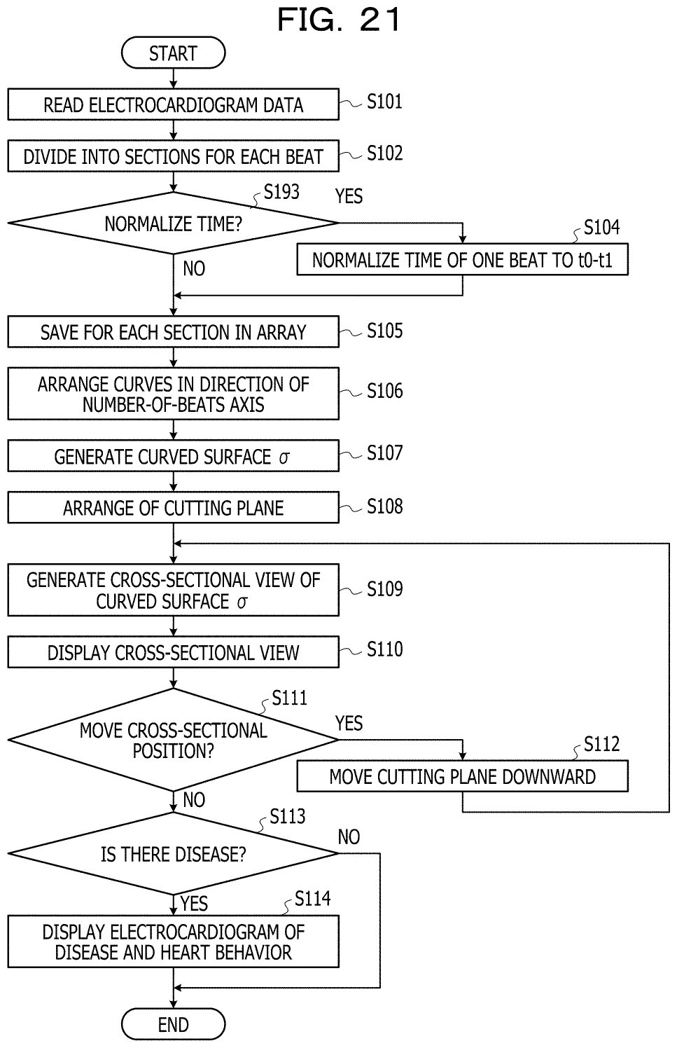

FIG. 21 is a flowchart illustrating an example of a procedure of the visualization processing. The processing illustrated in FIG. 21 will be described below according to step numbers.

[Step S101] The electrocardiogram visualization unit 130 reads the electrocardiogram data 121 from the storage unit 120.

[Step S102] The electrocardiogram visualization unit 130 divides the electrocardiogram data 121 into sections of each beat.

[Step S103] The electrocardiogram visualization unit 130 determines whether or not to normalize the time of one beat. For example, the electrocardiogram visualization unit 130 displays a message inquiring whether or not to normalize. When an input for performing normalization is made in response to the message, the electrocardiogram visualization unit 130 determines that the normalization is performed. Further, when an input for not performing the normalization is made, the electrocardiogram visualization unit 130 determines that the normalization is not performed. In a case where the normalization is performed, the electrocardiogram visualization unit 130 causes the processing to proceed to step S104. Further, in a case where the normalization is not performed, the electrocardiogram visualization unit 130 causes the processing to proceed to step S105.

[Step S104] The electrocardiogram visualization unit 130 normalizes the time of one beat to the time of t0-t1. The time of t0-t1 is, for example, an average time of one beat during a period in which a normal electrocardiogram appears.

[Step S105] The electrocardiogram visualization unit 130 stores the electrocardiogram data for each section in an array. For example, the electrocardiogram visualization unit 130 defines a two-dimensional array. In the electrocardiogram visualization unit 130, an order of the section (number of beats) is set in a first element number of the two-dimensional array. An identification number of the potential measured in the section is set in a second element number of the two-dimensional array. Then, a set of the potential measured in the corresponding section and an elapsed time from the start time of the corresponding section to the measurement time of the potential is set as a value of the array.

[Step S106] The electrocardiogram visualization unit 130 arranges a plurality of curves indicating an electrocardiogram for each section of one beat in the order of the beats in the direction of the number-of-beats axis in the three-dimensional coordinate system.

[Step S107] The electrocardiogram visualization unit 130 generates a curved surface a including the plurality of arranged curves indicating the electrocardiogram for the respective sections. The curved surface a is, for example, an NURBS curved surface.

[Step S108] The electrocardiogram visualization unit 130 sets the cutting plane on the upper side of the three-dimensional coordinate system (in a positive direction of potential). For example, the electrocardiogram visualization unit 130 sets a plane that passes through the maximum value of the potential indicated in the electrocardiogram data 121 and is parallel to the time axis and the number-of-beats axis, as an initial state of the cutting plane.

[Step S109] The electrocardiogram visualization unit 130 generates a cross-sectional view when the curved surface a is cut by the cutting plane.

[Step S110] The electrocardiogram visualization unit 130 displays the cross-sectional view on the monitor 21.

[Step S111] The electrocardiogram visualization unit 130 determines whether or not to move the cross-sectional position. For example, in a case where an automatic movement of the cross-sectional position is designated in advance, the electrocardiogram visualization unit 130 determines to move the cross-sectional position until the cutting plane reaches a lower limit unless an instruction to stop the movement is input. Further, when the automatic movement is not designated, the electrocardiogram visualization unit 130 waits for the movement instruction. When the movement instruction is input, the electrocardiogram visualization unit 130 determines to move the cross-sectional position. When the instruction to stop the movement is input, the electrocardiogram visualization unit 130 determines to stop the movement. In a case of moving the cross-sectional position, the electrocardiogram visualization unit 130 causes the processing to proceed to step S112. Further, in a case of stopping the movement of the cross-sectional position, the electrocardiogram visualization unit 130 causes the processing to proceed to step S113.

[Step S112] The electrocardiogram visualization unit 130 moves the cutting plane to the lower side by a predetermined amount. Thereafter, the electrocardiogram visualization unit 130 causes the processing to proceed to step S109.

[Step S113] The electrocardiogram visualization unit 130 causes the processing to branch depending on whether or not there is a disease based on the cross-sectional view. For example, when there is an input indicating that a disease is detected, the electrocardiogram visualization unit 130 causes the processing to proceed to step S114. Further, when there is an input indicating that there is no disease, the electrocardiogram visualization unit 130 ends the visualization processing.

[Step S114] The electrocardiogram visualization unit 130 displays an electrocardiogram during a period in which a disease appears and a cross-sectional view at that time. Further, the heart simulation unit 140 simulates the behavior of the heart when the symptom of the disease appears based on the electrocardiogram data during a period in which the disease appears, and reproduces the behavior by a heart model. The heart simulation unit 140 displays the behavior of the heart reproduced by the heart model on the monitor 21.

In this manner, by displaying the cross-sectional view based on the electrocardiogram, it is possible to determine the presence or absence of a disease and the type of the disease by determining the cross-sectional shape. Moreover, when a disease is found, it is possible to reproduce the behavior of the heart by a three-dimensional heart model in addition to the electrocardiogram and the cross-sectional view at that time.

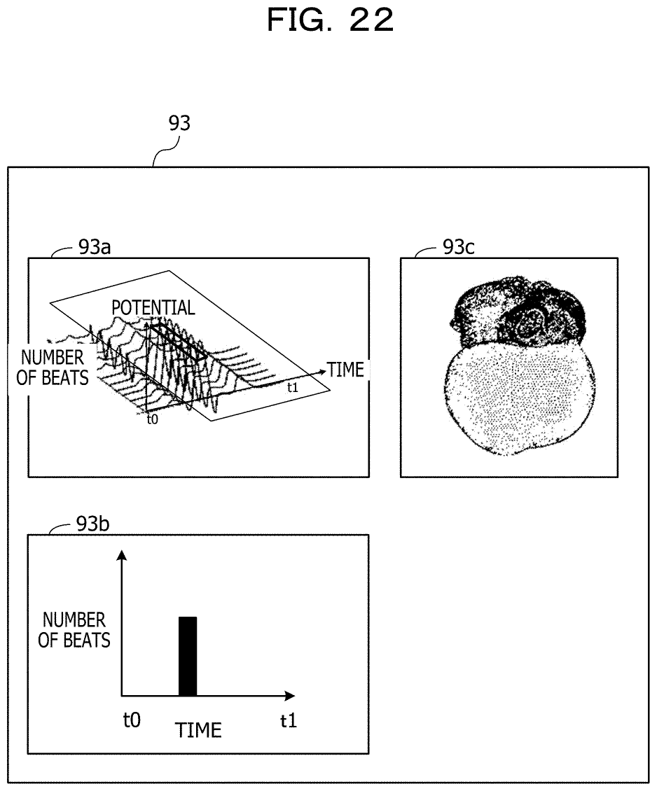

FIG. 22 illustrates an example of a visualization screen. A visualization screen 93 includes an electrocardiogram display unit 93a, a cross section display unit 93b, and a heart behavior display unit 93c.

The electrocardiogram display unit 93a displays an electrocardiogram arranged for each section of one beat. For example, based on electrocardiogram data in a period designated as a period in which the influence of the disease appears among long-term electrocardiogram data of 24 hours or longer, an electrocardiogram in this period is displayed on the electrocardiogram display unit 93a. Further, the electrocardiogram display unit 93a may also display the cutting plane that is used for generating the cross-sectional view displayed on the cross section display unit 93b.

The electrocardiogram display unit 93a may display the curved surface a including the plurality of curves indicating the electrocardiogram. In this case, the electrocardiogram visualization unit 130 colors, for example, the region, in the curved surface a, corresponding to intervals (PR interval, QRS interval, RR interval, and QT interval) of each of the PQRST waves during each beat according to a preset color map. The electrocardiogram visualization unit 130 performs, for example, coloring to emphasize a region having a long time interval. Further, the electrocardiogram visualization unit 130 may also display, for a QRS magnitude, a magnitude of the amplitude in an easily understandable manner by a difference in color.

For example, the electrocardiogram display unit 93a holds a color map in which colors corresponding to the magnitudes of the gradients are defined. In the color map, for example, a color change corresponding to a gradient from a minimum gradient value to a maximum gradient value is designated. The electrocardiogram display unit 93a obtains the gradients in the sections between the individual PQRST waves. The electrocardiogram display unit 93a allocates the colors of the minimum value and the maximum value of the color map to the minimum value and the maximum value of the gradient of the section. Then, the electrocardiogram display unit 93a allocates colors between the minimum value and the maximum value of the color map to the range from the minimum value to the maximum value of the gradient of the section. Further, the electrocardiogram display unit 93a colors the curved surface a with a color corresponding to the gradient.

By coloring the curved surface σ with a color corresponding to the gradient in this manner, when a beat in a state different from the beat state that has occurred so far appears in the electrocardiogram, it may be identified by the change in the color of the curved surface σ.

The cross section display unit 93b displays a cross-sectional view of the curved surface generated based on the electrocardiogram. For example, the cross section display unit 93b displays a cross-sectional view when the curved surface is cut by a cutting plane that may clearly represent the feature of the disease.

The heart behavior display unit 93c displays a reproduced image of the behavior of the heart reproduced based on the electrocardiogram data during a period in which the disease appears, that reflects the time-series change in the shape of the three-dimensional heart model. For example, when the number of beats (which number of beats) for which the behavior of the heart is to be reproduced is designated for the electrocardiogram display unit 93a or the cross section display unit 93b, the electrocardiogram visualization unit 130 transmits electrocardiogram data corresponding to the number of beats to the heart simulation unit 140. The heart simulation unit 140 executes simulation of the behavior of the heart based on the received electrocardiogram data, and reproduces the behavior of the heart by the heart model. When the number of beats in which the influence of the disease appears is designated, the heart that moves unnaturally due to the influence of the disease is visually displayed.

In this way, it is possible to easily determine the presence or absence of the disease based on the electrocardiogram. That is, the feature of the disease may be displayed in an easily understandable manner by the cross-sectional view of the curved surface including the plurality of curves indicating the electrocardiogram arranged separately for each beat. In addition, by normalizing the time of the electrocardiogram for one beat, the influence of the change in heart rate may be reduced, and the influence of the disease may be more strongly represented in the cross-sectional view.

Note that in a case where the doctor fails to find a disease when the time of the electrocardiogram for one beat is normalized and the visualization processing is executed, for example, the doctor causes the visualization system 100 to execute the visualization processing without normalization. By not performing the normalization, the difference in an actual cycle of each beat is displayed in an easily understandable manner. As a result, it becomes easy to find a disease that is difficult to find when normalized, such as bradycardia or tachycardia, by not performing the normalization. As described above, since it is possible to select the presence or absence of normalization, it is possible to find out more diseases without overlooking the diseases.

[Other Embodiments]

In the second embodiment, the cutting plane is moved from the upper side to the lower side, but the electrocardiogram visualization unit 130 may conversely move the cutting plane from the lower side to the upper side.

Further, the electrocardiogram visualization unit 130 may generate the cutting plane by inclining the cutting plane with respect to the time axis of the three-dimensional coordinate system. Depending on the disease, there may be a case where the influence of the disease appearing in the electrocardiogram becomes clearer in the cross-sectional view when the cutting plane is inclined with respect to the time axis. In a case of diagnosing the presence or absence of a disease, the doctor sets the inclination of the cutting plane for the visualization system 100. Thereby, the visualization system 100 may display the cross section by the cutting plane inclined with respect to the time axis.

While the embodiments are exemplified above, the configuration of each unit described in the embodiments may be replaced with another configuration having substantially the same function. Further, any other constituents or processes may be added. Moreover, any two or more of the configurations (features) described in the embodiments above may be combined with each other.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable storage medium for storing a visualization program which causes a processor to perform processing, the processing comprising:
arranging, based on electrocardiogram data that indicates an electrocardiogram obtained by sensing from a patient in a predetermined period, a plurality of curves in time series in an axial direction perpendicular to a time axis and a potential axis of the electrocardiogram, each of the plurality of curves indicates a partial electrocardiogram corresponding to a respective beat from among the electrocardiogram indicated in the electrocardiogram data;
generating a curved surface that includes the plurality of arranged curves;
generating a plane that intersects the curved surface; and
outputting a figure based on an intersection line between the curved surface and the plane.

2. The visualization program according to claim 1,
wherein, in displaying of the figure, a cross-sectional view that indicates a cross section obtained by cutting a region that has a lower potential than the curved surface by the plane is output, based on the intersection line between the curved surface and the plane.

3. The visualization program according to claim 1,
wherein, in setting of the plane, a position of the plane is moved parallel to a direction of the potential axis of the electrocardiogram, and
in the displaying, every time the plane moves, the figure based on the intersection line between the curved surface and the plane is output.

4. The visualization program according to claim 1,
wherein, in the arranging of the plurality of curves, a time taken for one beat is normalized so that a time taken for one beat indicated in the electrocardiogram of each beat is a predetermined time and the plurality of curves that indicate the electrocardiogram of each beat are arranged.

5. A computer-based visualization method comprising:
arranging, based on electrocardiogram data that indicates an electrocardiogram obtained by sensing from a patient in a predetermined period, a plurality of curves in time series in an axial direction perpendicular to a time axis and a potential axis of the electrocardiogram, each of the plurality of curves indicates a partial electrocardiogram corresponding to a respective beat from among the electrocardiogram indicated in the electrocardiogram data;
generating a curved surface that includes the plurality of arranged curves;
generating a plane that intersects the curved surface; and
outputting a figure based on an intersection line between the curved surface and the plane.

6. A visualization system comprising:
a memory; and
processor circuitry coupled to the memory, the processor circuitry being configured to perform processing, the processing including:
arranging, based on electrocardiogram data that indicates an electrocardiogram obtained by sensing from a patient in a predetermined period, a plurality of curves in time series in an axial direction perpendicular to a time axis and a potential axis of the electrocardiogram, each of the plurality of curves indicates a partial electrocardiogram corresponding to a respective beat from among the electrocardiogram indicated in the electrocardiogram data;
generating a curved surface that includes the plurality of arranged curves;
generating a plane that intersects the curved surface; and
outputting a figure based on an intersection line between the curved surface and the plane.
